(19) United States
(12) Patent Application Publication
(10) Pub. No.: US 2018/0322590 A1
(43) Pub. Date: Nov. 8, 2018

(54) INTEGRATED AGRICULTURAL TESTING AND OPTIMIZATION APPARATUSES AND METHODS

(71) Applicants: NARAYAN SUNDARARAJAN, Palo Alto, CA (US); GRACE M. CREDO, San Mateo, CA (US); TARA K. THIMMANAIK, Vancouver, WA (US); KAZI I. HUQUE, Portland, OR (US); SRINIVAS B. GARUDACHAR, Bangalore (IN); KATALIN K. BARTFAI-WALCOTT, El Dorado Hills, CA (US); KHURSHADUZZAMAN RAZIB, Dhaka (BD); FAHIM HASNAEEN, Dhaka (BD); NUZHAT BINTE ARIF, Dhaka (BD)

(72) Inventors: NARAYAN SUNDARARAJAN, Palo Alto, CA (US); GRACE M. CREDO, San Mateo, CA (US); TARA K. THIMMANAIK, Vancouver, WA (US); KAZI I. HUQUE, Portland, OR (US); SRINIVAS B. GARUDACHAR, Bangalore (IN); KATALIN K. BARTFAI-WALCOTT, El Dorado Hills, CA (US); KHURSHADUZZAMAN RAZIB, Dhaka (BD); FAHIM HASNAEEN, Dhaka (BD); NUZHAT BINTE ARIF, Dhaka (BD)

(21) Appl. No.: 15/862,247

(22) Filed: Jan. 4, 2018

Related U.S. Application Data
(60) Provisional application No. 62/500,954, filed on May 3, 2017.

(51) Int. Cl. G06Q 50/02; G01N 33/24; G06Q 10/06; G06Q 30/06

(52) U.S. Cl. CPC G06Q 50/02; G01N 33/24; G01N 21/25; G06Q 30/0613; G01N 2033/245; G06Q 10/06313

(57) ABSTRACT

Apparatuses, methods and storage media associated with agricultural testing and optimization are disclosed herein. In embodiments, an apparatus for performing agricultural testing and optimization may comprise a cavity to receive a container of soil nutrient solution sample of a location in an agricultural region; one or more sensors to collect sensor data from the soil nutrient solution sample; and one or more agricultural testing and optimization applications to perform agricultural testing and optimization for the location, based at least in part on the sensor data collected from the soil nutrient solution sample. Other embodiments may be disclosed or claimed.

### RELATED APPLICATION

This application is a non-provisional application of provisional application U.S. 62/500,954, filed May 3, 2017, entitled “Integrated Agricultural Testing and Optimization Apparatuses and Methods,” the specification and drawings of which are hereby fully incorporated by reference.

### TECHNICAL FIELD

The present disclosure relates to the fields of computing and agriculture, in particular, to apparatuses, methods and storage media associated with agricultural testing and optimization.

### BACKGROUND

The background description provided herein is for the purpose of generally presenting the context of the disclosure. Unless otherwise indicated herein, the materials described in this section are not prior art to the claims in this application and are not admitted to be prior art by inclusion in this section.

According to the United Nations, the world's farmers will need to feed 9.6 billion people by 2050, and that will require a 70% increase in agricultural production. Agriculture is a $3 trillion/year industry that is very sophisticated in some countries and lacking basic information in others. Soil nutrient imbalance (too much or too little of certain chemical compounds in soil) is known to reduce crop yield and quality, resulting in wasted use of fertilizer and water on a global scale. One approach to maximizing crop yield is to optimize the numerous nutrients found in soil; however, soil nutrients are not monitored in many parts of the world largely due to lack of affordable, easy-to-use sensing tools at or near the field.

### BRIEF DESCRIPTION OF THE DRAWINGS

Embodiments will be readily understood by the following detailed description in conjunction with the accompanying drawings. To facilitate this description, like reference numerals designate like structural elements. Embodiments are illustrated by way of example, and not by way of limitation, in the figures of the accompanying drawings.

FIG. 1 illustrates an integrated agricultural testing and optimization system, in accordance with various embodiments.

FIG. 2 illustrates a cross section view of a portable integrated agricultural testing and optimization device, and its associated soil testing tube, in accordance with various embodiments.

FIG. 3 illustrates a component view of a portable integrated agricultural testing and optimization device, in accordance with various embodiments.

FIG. 4 illustrates a software view of a portable integrated agricultural testing and optimization device, in accordance with various embodiments.

FIG. 5 illustrates a block diagram view for seed recommendation, in accordance with various embodiments.

FIG. 6 illustrates a block diagram view for soil management, in accordance with various embodiments.

FIG. 7 illustrates a block diagram view for pesticide/additive management, in accordance with various embodiments.

FIG. 8 illustrates a block diagram view for harvest and sale management, in accordance with various embodiments.

FIG. 9 illustrates an architecture view of a computing apparatus suitable for use as a portable integrated agricultural testing and optimization device or a supporting cloud server, in accordance with various embodiments.

FIG. 10 illustrates an example storage medium with instructions configured to enable a portable integrated agricultural testing and optimization device, or a supporting cloud server to practice respective aspects of the present disclosure, in accordance with various embodiments.

FIG. 11 illustrates an example integrated chip containing an array of sensors, in accordance with various embodiments.

FIG. 12 illustrates an example implementation of an electrochemical sensor array, in accordance with various embodiments.

### DETAILED DESCRIPTION

Apparatuses, methods and storage media associated with an integrated agricultural testing and optimization system are disclosed herein. In embodiments, an integrated agricultural testing and optimization system may include portable integrated agricultural testing and optimization devices, designed for field use, and cloud services in support of the portable integrated agricultural testing and optimization devices. The portable integrated agricultural testing and optimization devices of the present disclosure may target the large numbers of small farm holders who currently are not able to collect and use information about their soil. Current approaches to soil nutrient measurement rely on large centralized facilities that can be too expensive for small farmers to access, typical instrumentation is too large/expensive and not portable, the wet chemistry test process is time and labor intensive and even portable chemistry kits suffer from hard to interpret results (color cards). The portable integrated agricultural testing and optimization devices of the present disclosure may be configured to measure absorbance of test solutions after soil has been treated, individually to extract and label nutrients in water. Using a local software database and/or a cloud database that connects to one of these portable integrated agricultural testing and optimization devices, a farmer may get a recommendation on how much fertilizer to apply to their soil and crop. In embodiments, the portable integrated agricultural testing and optimization devices also include other applications to provide recommendation on what type of seed type of to plant, how to deal with pests, and/or when to harvest and what price to sell. The portable integrated agricultural testing and optimization system of the present disclosure may be used by the farmers, social entrepreneurs, government or non-governmental officials, or their consultants, in the fields or in forward/regional laboratories or offices near the fields. Thus, the portable integrated agricultural testing and optimization system of the present disclosure may provide the benefit of automating both the measurement stage and the connection between the data and the fertilizer recommendation.

In the description to follow, reference is made to the accompanying drawings which form a part hereof wherein like numerals designate like parts throughout, and in which is shown by way of illustration embodiments that may be practiced. It is to be understood that other embodiments may be utilized and structural or logical changes may be made without departing from the scope of the present disclosure. Therefore, the following detailed description is not to be taken in a limiting sense, and the scope of embodiments is defined by the appended claims and their equivalents.

Operations of various methods may be described as multiple discrete actions or operations in turn, in a manner that is most helpful in understanding the claimed subject matter. However, the order of description should not be construed as to imply that these operations are necessarily order dependent. In particular, these operations may not be performed in the order of presentation. Operations described may be performed in a different order than the described embodiments. Various additional operations may be performed and/or described operations may be omitted, split or combined in additional embodiments.

For the purposes of the present disclosure, the phrase “A and/or B” means (A), (B), or (A and B). For the purposes of the present disclosure, the phrase “A, B, and/or C” means (A), (B), (C), (A and B), (A and C), (B and C), or (A, B and C).

The description may use the phrases “in an embodiment,” or “in embodiments,” which may each refer to one or more of the same or different embodiments. Furthermore, the terms “comprising,” “including,” “having,” and the like, as used with respect to embodiments of the present disclosure, are synonymous.

As used hereinafter, including the claims, the term “module” may refer to, be part of, or include an Application Specific Integrated Circuit (ASIC), an electronic circuit, a programmable combinatorial circuit (such as a field programmable gate array (FPGA)), a processor (shared, dedicated, or group) and/or memory (shared, dedicated, or group) that execute one or more software or firmware programs generated from a plurality of programming instructions, and/or other suitable components that provide the described functionality.

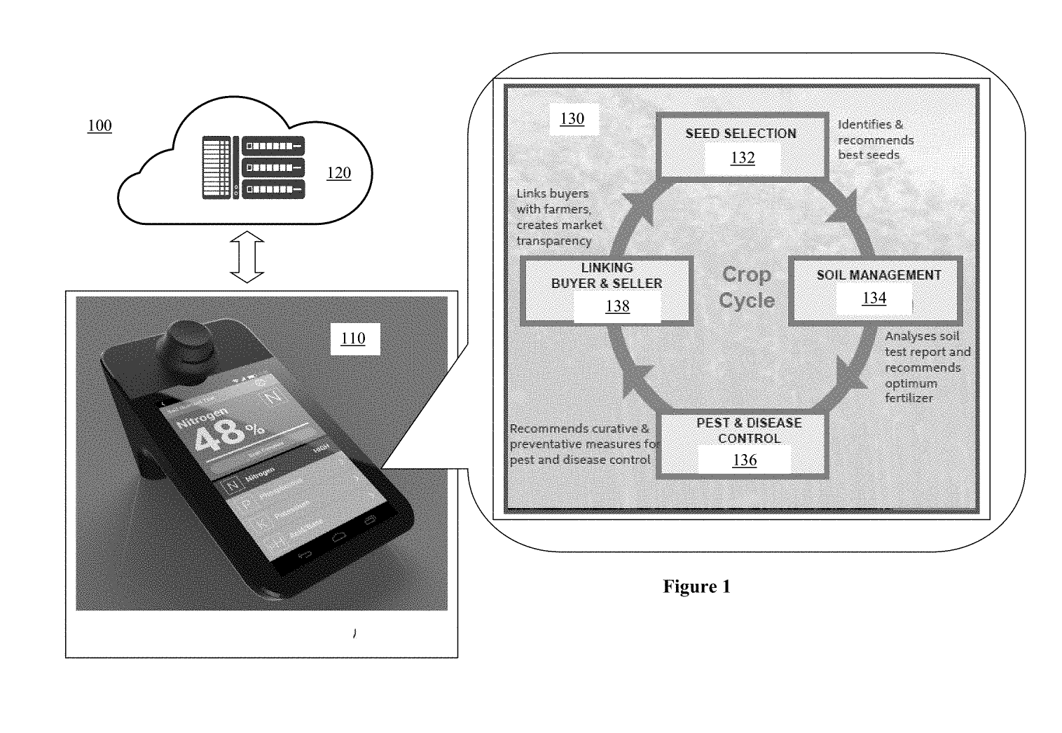

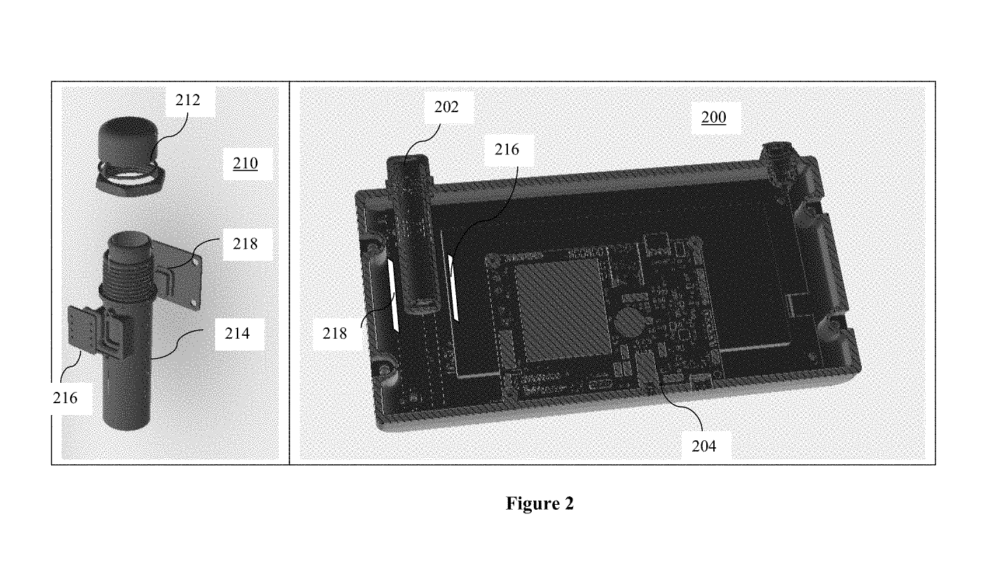

Referring now FIG. 1, wherein an overview of the integrated agricultural testing and optimization system of the present disclosure, according to various embodiments, is shown. As illustrated, in embodiments, integrated agricultural testing and optimization system 100 may include a number of portable integrated agricultural testing and optimization devices 110 (one shown), designed for field use, and cloud servers 120 configured to support portable integrated agricultural testing and optimization devices 110. In embodiments, one or more cloud servers 120 may be located in a fog network. Thus cloud servers 120 may also be referred to as cloud/fog servers 120. Each portable integrated agricultural testing and optimization device 110 may be configured to accept and analyze soils samples (at the not visible side; see FIG. 2). Further, each portable integrated agricultural testing and optimization device 110 may be configured with a number of crop cycle related applications 130, e.g., seed selection application 132, soil management application 134, pest & disease control application 136, and/or buyer and seller linking application 138. In embodiments, seed selection application 132 may be configured to assist a farmer, a social entrepreneur, or their consultant(s) (hereinafter, simply referred generically as “the farmer”) in selecting seed/crop for the soil at a location of an agricultural region. Soil management application 134 may be configured to assist the farmer to manage the nutrient of the soil at the location of the agricultural region. Pest & disease control application 136 may be configured to assist the farmer in managing pest & disease in the soil at the location of the agricultural region, while the seed germinates and the crop grows. Buyer and seller linking application 138 may be configured to assist the farmer in managing the harvesting and selling of the crops. Resultantly, farmers (including small farmers and/or famers in underdeveloped regions of the world) may have access to advanced technology to assist them in seed/crop selection, soil management, pest and disease control, harvesting and selling, improving their productivity, profitability, and/or sustainability. In alternate embodiments, crop cycle related applications 130 may include more or less applications.

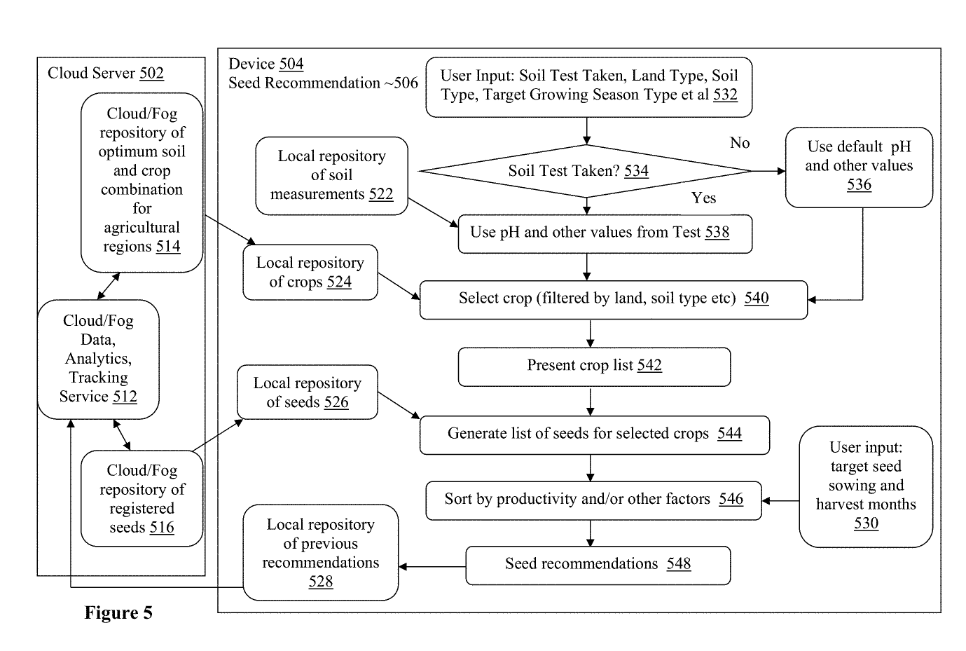

Cloud/fog servers 120, as will be described in more detail below with references to FIGS. 5-8, may include a number of repositories of agricultural data for a plurality of agricultural regions, and a plurality of cloud/fog data analytics tracking and services to provide agricultural testing and optimization support services to a plurality of portable integrated agricultural testing and optimization devices 110 to operate in a plurality of locations of the plurality of agricultural regions to provide in field agricultural testing and optimization. In embodiments, cloud/fog servers 120 may include a number of storage devices to store the plurality of repositories, and processors and memory arrangements to host and operate the cloud/fog data analytics tracking and services. Further, cloud/fog servers 120 may include a plurality of communication interfaces to communicate with the portable integrated agricultural testing and optimization devices 110 operating in the field at various locations of the various agricultural regions.

FIG. 2 illustrate a cross section view of a portable integrated agricultural testing and optimization device and its associated soil testing tube, in accordance with various embodiments. As illustrated, portable integrated agricultural testing and optimization device 200, which may be portable integrated agricultural testing and optimization device 100 of FIG. 1, may include circuit board 204 having a number of electronic components, e.g., processor, memory and so forth. Further, portable integrated agricultural testing and optimization device 200 may include an integrated cavity 202 configured to receive a container 210 of soil nutrient solution sample for nutrient and other analyses. For the illustrated embodiments, container 210 is cylindrical, elongated, tubular in shape, and integrated cavity 202 is complementarily shaped, i.e., cylindrical and elongated, to receive container tube 210. Additionally, container tube 210 may have an associated cap 212. In alternate embodiments, container 210 and integrated cavity 202 may be of other geometry sizes and/or shapes. In embodiments, if portable integrated agricultural testing and optimization device 200 is used by social entrepreneurs or consultants for multiple farmers, e.g., in forward/regional laboratories/offices, the soil samples may be geo-tagged to identify the associated fields/farmers of the soil samples.

Still further, circuit board 204 may include various sensors to facilitate the nutrient and other analyses of the soil nutrient solution sample. For the illustrated embodiments, circuit board 204 may include a number of Red/Green/Blue (RGB) color sensors 216 and associated light-emitting diode (LED) white light source 218. LED white light source 218 may be configured to illuminate the soil nutrient solution sample within the container 210, and RGB color sensors 216 may be configured to sense the color of the soil nutrient solution sample in the container 210, based on light impact onto color sensors 216 after passing through the soil nutrient solution sample. Color sensors 216 may be configured to sense the amount of nutrients nitrogen (N), phosphorous (P), and potassium (K) as well as the solution characteristic pH (concentration of H ions) in the soil sample. For these light sensing based embodiments, cavity 202 may be further configured to shield and protect the soil nutrient solution sample within the container 210 from external light. For these embodiments, the body of portable integrated agricultural testing and optimization devices 200 may be configured to substantially seal the interior space housing the internal components, in particular, the space within cavity 202 from external light.

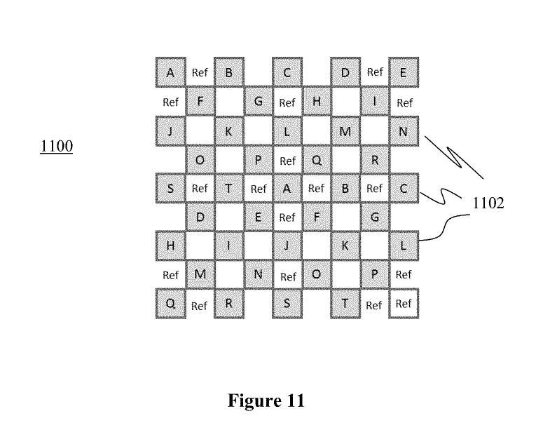

In alternate embodiments, other forms of sensing, such as electrochemical or silicon photonic sensing, may be used, in lieu of optical color sensing. FIG. 11 illustrates an example integrated chip containing an array of sensors, in accordance with various embodiments. As shown, example integrated chip 1100 contains an array of sensor elements 1102. The array of sensor elements 1102 may be configured to cover as many nutrients and solution characteristics (pH, conductivity) as the number of sensor elements available. The array 1100 can have one sensor element 1102 per nutrient or more (2 or 3 or more per nutrient) to be redundant. In embodiments, array 1100 may also include reference sensor element(s) (REF) to establish a baseline response as a function of clean solution, manufacturing variations, and/or temperature fluctuations.

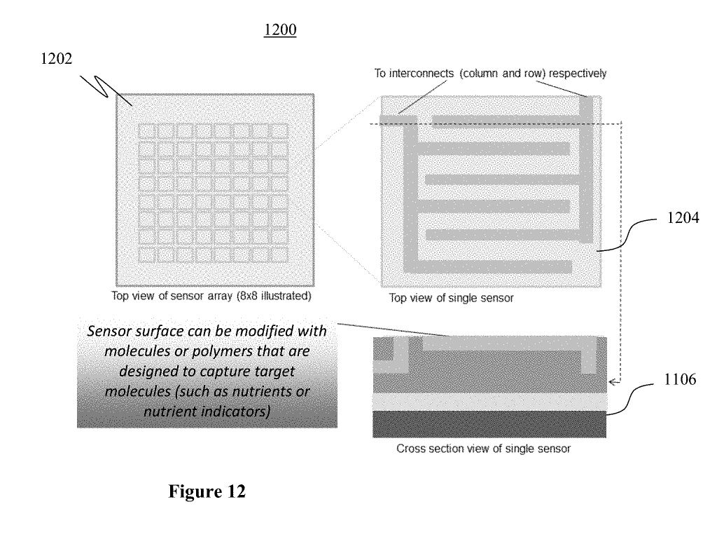

FIG. 12 illustrates an example implementation of an electrochemical sensor array, in accordance with various embodiments. Portion 1202 of FIG. 12 illustrates a top view of the example 8×8 implementation. Portion 1204 of FIG. 12 illustrates a top view of a single sensor element of the 8×8 implementation. And portion 1206 of FIG. 12 illustrates a cross section view of the single sensor element illustrated in portion 1204. As shown in the top and cross section views of the single sensor element, each sensor element may have electrodes to interconnect the sensor element with signal lines of the sensor array. The electrode may comprise gold, platinum, carbon, or diamond. The surface of the electrode may be bare metal or metal oxide or coated with a dielectric materials such as silicon oxide. Further, each sensor element could be modified with a target-specific molecule, peptide or polymer. In embodiments, the target-specific peptide or polymer could be redox-neutral. Instead the solution could contain free molecules that are easily oxidized and reduced such as ferrocene or methylene blue molecules. When the peptide/polymer binds to the target, it could fold or configure in such a way that the electrode surface is better insulated (less free space for molecules to diffuse, or thicker polymer layer), decreasing the signal level. Further, the target-specific peptide or polymer could be terminated with a molecule that could easily be oxidized or reduced, such as ferrocene or methylene blue. When the peptide/polymer binds to the target, it could fold or configure in such a way that the Fc or MB molecules are closer to the electrode surface, increasing the signal.

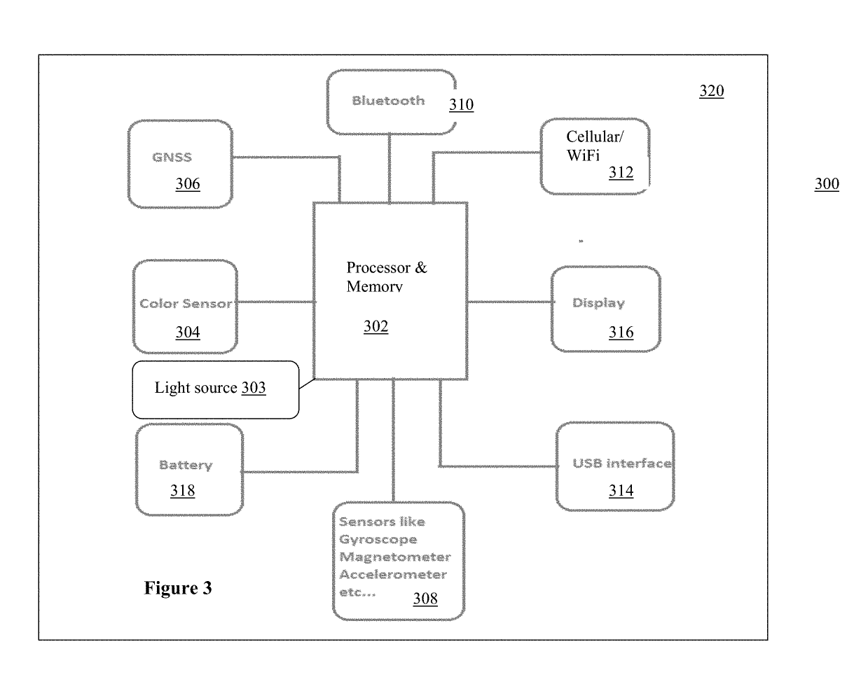

FIG. 3 illustrates a component view of a portable integrated agricultural testing and optimization device, in accordance with various embodiments. As illustrated, portable integrated agricultural testing and optimization device 300, which may be portable integrated agricultural testing and optimization device 110 of FIG. 1 or 200 of FIG. 2, may include a number of electronic components 302-318, housed by body 320. Body 320 may have dimensions and shape configured for portability of integrated agricultural testing and optimization device 300.

In embodiments, electronic components 302-318 may include processor and memory arrangement 302 configured to store and execute programming instructions configured to implement the various crop cycle related applications, e.g., seed selection application 132, soil management application 134, pest and disease control application 136 and buyer and seller linking application 138. In embodiments, electronic components 302-318 may further include color sensors 304 configured to sense colors of a soil nutrient solution sample, and light source 303 for providing light to the soil nutrient solution sample. As described earlier, in alternate embodiments, in lieu of color sensors 304 and light source 303, electrochemical or other sensors may be employed instead. In embodiments, electronic components 302-318 may further include global network satellite system (GNSS) sensors 306 configured to sense location of device 300, and other sensors 308 like gyroscope, magnetometer, accelerometer etc. to collect and provide various sensor data for the control and operation of portable integrated agricultural testing and optimization device 300. In embodiments, electronic components 302-318 may further include various interface/communication components, such as Bluetooth® or other near field communication interface 310, Cellular and/or WiFi communication interface 312, and Universal Serial Bus (USB) and/or other interconnect interface 314. Interface 314 may be configured to receive user inputs from various input device, including, but are not limited, (detachable) keyboard and/or cursor control devices. In embodiments, electronic components 302-318 may further include a display 316, e.g., a touchscreen for receiving input and rendering outputs. In embodiments, electronic components 302-318 may further include battery 318 to provide power to portable integrated agricultural testing and optimization device 300.

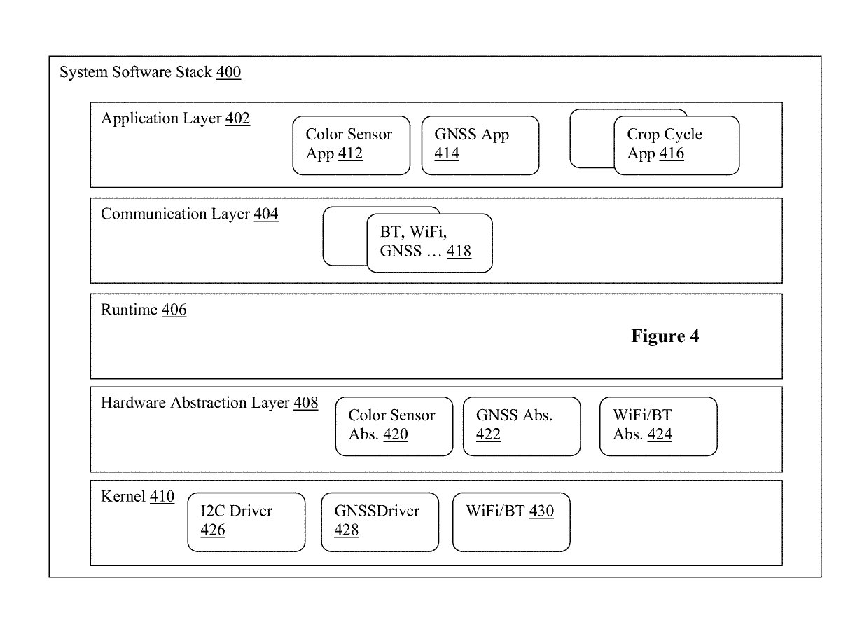

FIG. 4 illustrates a software view of a portable integrated agricultural testing and optimization device, in accordance with various embodiments. As illustrated, a portable integrated agricultural testing and optimization device, which may be portable integrated agricultural testing and optimization device 110 of FIG. 1, 200 of FIG. 2 or 300 of FIG. 3, may include a number of system software layers 400. In embodiments, system software layers 400 may include application layer 402, communication layer 404, runtime layer 406, hardware abstraction layer 408 and kernel 410.

In embodiments, application layer 402 may include color sensor application 412 configured to process color sensor data to provide color readings of a soil nutrient solution sample, GNSS application 414 configured to process location data to provide geo-location of the portable integrated agricultural testing and optimization device, and various crop cycle applications 416. In embodiments, crop cycle applications 416 may include the earlier described seed selection application 132, soil management application 134, pest & disease control application 136, and buyer and/or seller linking application 138.

In embodiments, communication layer 404 may include various communication system services, e.g., for Bluetooth®, WiFi, Cellular, GNSS et al communications. Hardware abstraction layer 408 may include abstraction of color sensors, GNSS, WiFi, Bluetooth® et al hardware for the earlier described software applications and/or services. Kernel 410 may include various drivers, e.g., I2C, GNSS, WiFi, Bluetooth®, and so forth.

In embodiments, runtime layer 406 may include various conventional runtime facilities known in the art. In embodiments, hardware abstraction layer 408 may include various abstraction of hardware services, making such services easier to be used by various applications 412-416 of application layer 402, e.g., color sensor service abstraction 420, GNSS service abstraction 422, and WiFi/BT service abstraction 424. In embodiments, kernel 410 may include various hardware drivers configured to control/operate various hardware components of the portable integrated agricultural testing and optimization device, e.g., I2C driver 426, GNSS driver 428 and WiFi/BT driver 430.

FIG. 5 illustrates a block diagram view for seed recommendation, in accordance with various embodiments. As illustrated, on the cloud side, cloud server 502, which may be a server or a cluster of servers of cloud servers 120 of FIG. 1, may include cloud/fog data analytics tracking and services 512, cloud/fog repository 514 of optimum soil and crop combination for various agricultural regions, and cloud/fog repository 516 of registered seeds. Cloud/fog data analytics tracking and services 512 may be configured to maintain and update the optimum soil and crop combination data for various agricultural regions, and seed data in repository 514 and 516, and interact with various portable integrated agricultural testing and optimization devices (e.g., device 504) to provide support services, including but not limited to the provision of the relevant subset of these data to corresponding repository (e.g., repository 524 and 526) to various portable integrated agricultural testing and optimization devices (e.g., device 504).

Still referring to FIG. 5, portable integrated agricultural testing and optimization device 504 may be any one of portable integrated agricultural testing and optimization devices 110, 200, 300, or 400 of FIG. 1, 2, 3 or 4. Seed recommendation application 506 may be seed recommendation application 134 of FIG. 1. Seed recommendation application 506 on each device 504 may include repository 524 and 526. Repository 524 may be configured to store crop information suitable for the agricultural region where device 504 is being used. Repository 526 may be configured to store the seed information for growing such crops. Data in repository 524 and 526 may be downloaded from repository 514 and 516 in cloud server 502. Further, each device 504 may include local repository 522 of soil measurements, and local repository 528 of previous seed/crop recommendations.

Seed recommendation application 506 may be configured to receive, during operation, user inputs on soil test taken, land type, soil type, target growing season type, and so forth 532, and in response provide seed/crop recommendations. In embodiments, based on the input, seed recommendation application 506 may determine whether soil test was taken. If soil test was taken (e.g., by virtue of their availability in local repository 522 of soil measurements), seed recommendation application 506 may operate with the pH and other relevant values from the soil test 538. On the other hand, if soil test was not taken, seed recommendation application 506 may operate with default pH and other relevant values 536. On determining the pH and other values to be used, seed recommendation application 506 may select crops 540 from local repository 524 of crops, based on the pH and other relevant values. In embodiments, the crops selected may be filtered by land, soil type and so forth.

Then, seed recommendation application 506 may present the list of selected crops for user to select 542. On receipt of the user's selection(s), seed recommendation application 506 may generate a list of seeds for the selected crops, accessing local repository 526 of seeds. Next, seed recommendation application 506 may sort the selection by productivity and/or other factors 546. The sorting may be further based on user inputs on target seed sowing and harvest months 530. Thereafter, seed recommendation application 506 may recommend the seeds for growing the selected crops 548 (from the list of seeds, and/or in view of the further user inputs). In embodiments, the recommendations may be recorded in local repository 528, e.g., to take into consideration for future recommendations.

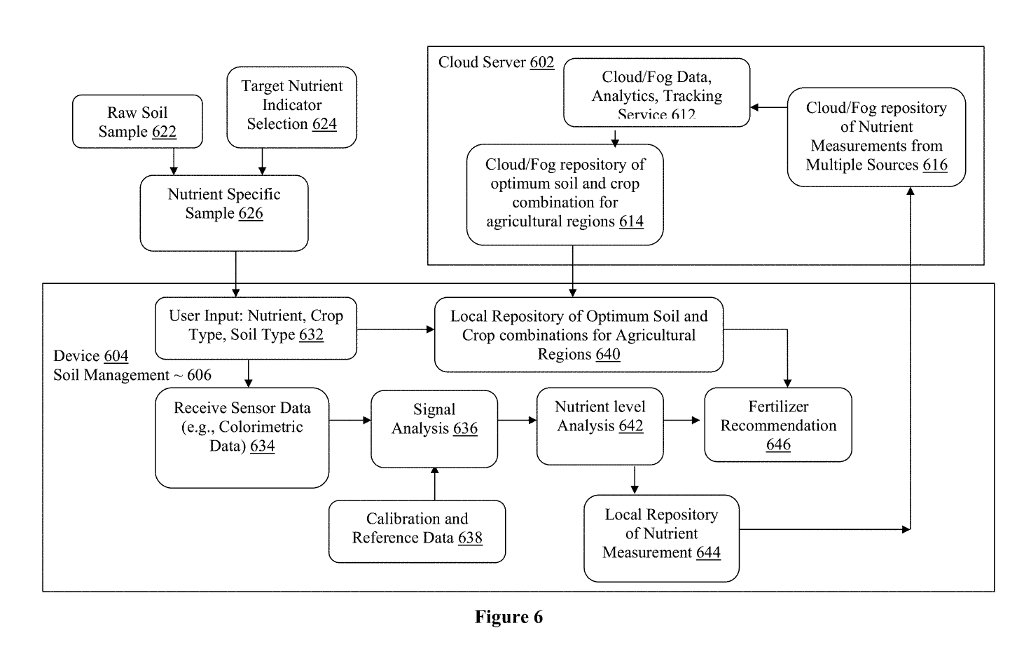

FIG. 6 illustrates a block diagram view for soil management, in accordance with various embodiments. As illustrated, on the cloud side, cloud server 602, which may be a server or a cluster of servers of cloud servers 120 of FIG. 1, may include cloud/fog data analytics tracking and services 612, cloud/fog repository 614 of optimum soil and crop combination for various agricultural regions, and cloud/fog repository 616 of nutrient measurements from multiple sources (e.g., other portable integrated agricultural testing and optimization devices). Cloud/fog data analytics tracking and services 612 may be configured to maintain and update the optimum soil and crop combination data for various agricultural regions, and nutrient measurements in repository 614 and 616, and provide the relevant subset of these data to corresponding repository (e.g., repository 640) in various portable integrated agricultural testing and optimization devices (e.g., device 604).

Still referring to FIG. 6, portable integrated agricultural testing and optimization device 604 may be any one of portable integrated agricultural testing and optimization devices 110, 200, 300, or 400 of FIG. 1, 2, 3 or 4. Soil management application 606 may be soil management application 134 of FIG. 1. Soil management application 606 may include repository 640 and 644. Repository 640 may be configured to store crop information suitable for the agricultural region where device 604 is being used. Data in repository 640 may be downloaded from repository 614 in cloud server 602. Further, each device 604 may include local repository 644 of nutrient measurements. In embodiments, nutrient measurement data in repository 644 may be uploaded to cloud/fog repository 616 of nutrient measurements, to contribute to the crowd sourced data accumulated therein, for use by cloud/fog data analytics tracking service 612 in providing its services to various portable integrated agricultural testing and optimization devices.

Soil management application 606 may be configured to receive, during operation, user inputs on nutrient, crop type, soil type, and so forth 632, and in response, provide soil management recommendations. In embodiments, nutrient input may be received from the nutrient specific sample 626 prepared by the user. Nutrient specific sample (i.e., earlier described soil nutrient solution sample) 626 may be prepared from raw soil sample 622 and target nutrient indicator selection 624 made by the user. In embodiments, a plurality of nutrients and other soil properties may be supported, including but are not limited to Nitrogen (N), Phosphorus (P), Potassium (K), Copper (Cu), Cobalt (Co), Iron (Fe), Zinc (Zn), Manganese (Mn), Calcium (Ca), Magnesium (Mg), Sulfur (S), Boron (B), Molybdenum (Mo), Chlorine (Cl), Nickel (Ni), Carbon (C), Aluminum (Al), alkalinity or acidity as measured in potential of hydrogen (pH), and so forth. In embodiments, on receipt of the user inputs, soil management application 606 may receive soil sample analysis outputs, e.g., colorimetric data output by RGB color sensors in response to sensing color of the soil nutrient solution sample input. Soil management application 606 then processes the sensed data (e.g., colorimetric, electrochemical, silicon photonics, and so forth) 634 and performs signal analysis 636. The analysis may be performed using calibration and/or reference data 638 provided to device 604. From the results of the signal analysis, soil management application 606 may perform nutrient level analysis 642. Based on the results of the nutrient level analysis 642, soil management application 606 may recommend a type and amount of fertilizer 646 to the user. In embodiments, the results of the nutrient level analysis 642 may be stored in local repository 644, and as described earlier, periodically uploaded to cloud server 602.

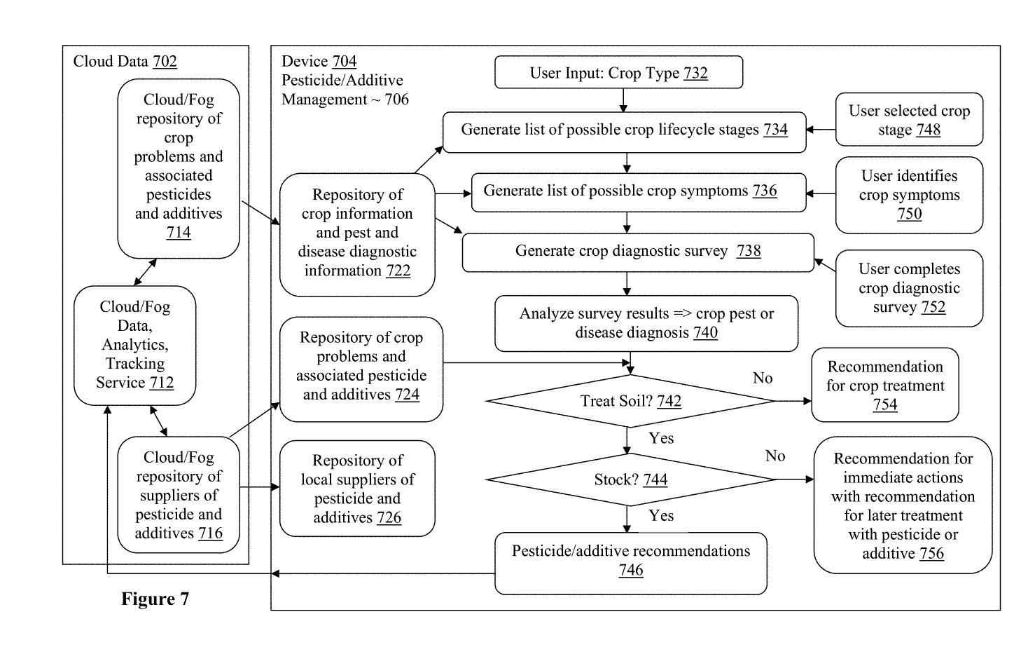

FIG. 7 illustrates a block diagram view for pesticide/additive management, in accordance with various embodiments. As illustrated, on the cloud side, cloud server 702, which may be a server or a cluster of servers of cloud servers 120 of FIG. 1, may include cloud/fog data analytics tracking and services 712, cloud/fog repository 714 of crop problems and associated pesticides and additives, and cloud/fog repository 716 of various suppliers of various pesticides and additives. Cloud/fog data analytics tracking and services 712 may be configured to maintain and update the crop problems and associated pesticides and additives data, and suppliers of various pesticides and additives in repository 714 and 716, and provide the relevant subset of these data to corresponding repository (e.g., repository 722, 724 and 726) in various portable integrated agricultural testing and optimization devices (e.g., device 704).

Still referring to FIG. 7, portable integrated agricultural testing and optimization device 704 may be any one of portable integrated agricultural testing and optimization devices 110, 200, 300, or 400 of FIG. 1, 2, 3 or 4. Pesticide/additive management application 706 may be pesticide/additive management application 136 of FIG. 1. In embodiments, pesticide/additive management application 706 on each device 704 may include repository 722, 724 and 726. Repository 722 may be configured to store crop information and pest and disease diagnostic information. Repository 724 may be configured to store crop problems and associated pesticide and additive treatment information. Repository 726 may be configured to store the local supplier of pesticides and additives. Data in repository 722, 724 and 726 may be downloaded from repository 714 and 716 in cloud server 702.

In embodiments, pesticide/additive management application 706 may be configured to receive, during operation, user inputs on crop type 732. Based on the input, pesticide/additive management application 706 may generate a list of possible crop lifecycle stages 734 for selection 748 by a user. The lifecycle stages 734 may be generated using crop and pesticide and disease data stored in repository 722. On receipt of the selection, pesticide/additive management application 706 may generate a list of possible crop symptoms 736 for identification 750 by a user. The crop symptoms 736 may be also generated using crop and pesticide and disease data stored in repository 722. On receipt of the identification, pesticide/additive management application 706 may generate a crop diagnostic survey 738 for completion 752 by a user. The crop diagnostic survey 738 may be also generated using crop and pesticide and disease data stored in repository 722.

On receipt of the completed survey, pesticide/additive management application 706 may analyze the survey results to provide a crop pesticide or disease diagnosis 740. Based on the diagnosis, pesticide/additive management application 706 may determine whether pesticide(s) or additive(s) are needed for treatment of the soil 742. If pesticide(s) or additive(s) are not needed for treatment of the soil, pesticide/additive management application 706 proceeds to recommend treatment for the crop 754. On the other hand, if pesticide(s) or additive(s) are needed for treatment of the soil, pesticide/additive management application 706 further determines whether the needed pesticide(s)/additive(s) are available in the local suppliers 744. If pesticide(s) or additive(s) needed for treatment of the soil are not available in local suppliers, pesticide/additive management application 706 proceeds to recommend immediate action, and later treatment with pesticide(s) and additive(s) 756. On the other hand, if pesticide(s) or additive(s) needed for treatment of the soil are available in local suppliers, pesticide/additive management application 706 recommends immediate treatment with pesticide(s) and additive(s) 746 from local suppliers. In embodiments, the recommendations are further reported to cloud/fog data analytics tracking and services 712 of cloud server 702.

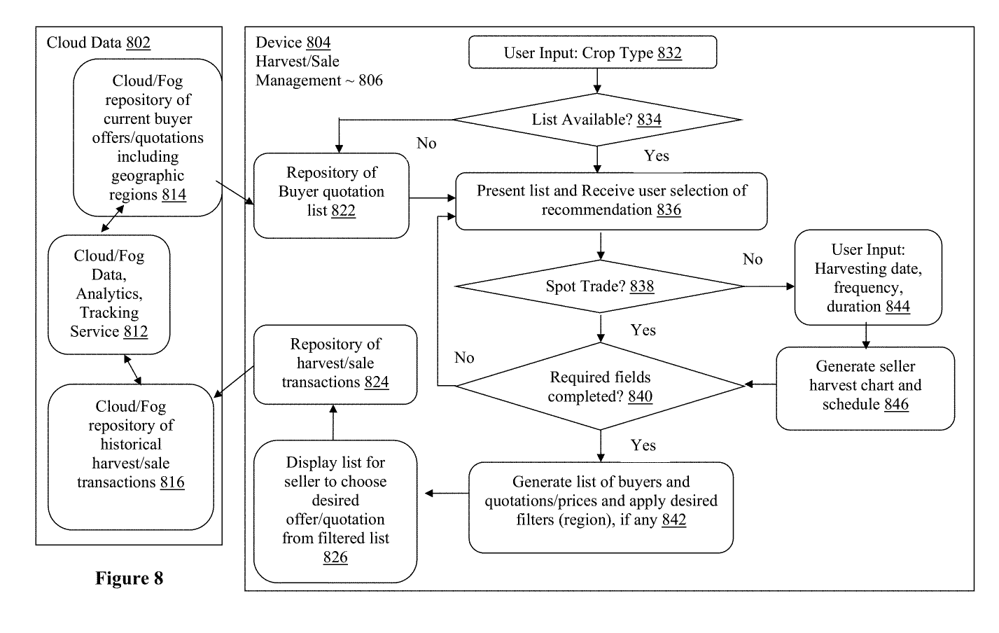

FIG. 8 illustrates a block diagram view for harvest and sale management, in accordance with various embodiments. As illustrated, on the cloud side, cloud server 802, which may be a server or a cluster of servers of cloud servers 120 of FIG. 1, may include cloud/fog data analytics tracking and services 812, cloud/fog repository 814 of current buyer offers/quotations for various crops (optionally, by geographic regions), and cloud/fog repository 816 of historical harvest/sale recommendations. Cloud/fog data analytics tracking and services 812 may be configured to maintain and update current buyer offers/quotations for various crops (and geographic region) data, and historical harvest/sale recommendations in repository 814 and 816, and provide the relevant subset of these data to corresponding repository (e.g., repository 822) in various portable integrated agricultural testing and optimization devices (e.g., device 804).

Still referring to FIG. 8, portable integrated agricultural testing and optimization device 804 may be any one of portable integrated agricultural testing and optimization devices 110, 200, 300, or 400 of FIG. 1, 2, 3 or 4. Harvest and sale management application 806 may be buyer and seller linking application 138 of FIG. 1. In embodiments, harvest and sale management application 806 on each device 804 may include repository 822, and 824. Repository 822 may be configured to store buyer quotation information. Repository 824 may be configured to store various harvest/sale transactions. Data in repository 822 may be downloaded from repository 814 in cloud server 802. Data in repository 824 may be periodically uploaded to repository 814 in cloud server 802.

In embodiments, harvest and sale management application 806 may be configured to receive, during operation, user inputs on crop type 832. Based on the input, harvest and sale management application 806 may determine whether a current list of buyer offers is available 834. If a current list of buyer offers are not available, harvest and sale management application 806 access repository 822 and refresh the list. On determining a current list is available or on refresh, harvest and sale management application 806 present the list for user selection, and receive the user's selection 836. On receipt of the user selection, harvest and sale management application 806 further determines whether the selected offer is a spot trade 834. If the selected offer is not a spot trade, harvest and sale management application 806 further solicits and receive user inputs on harvesting date, frequency and duration of harvest, and so forth 844. Next, harvest and sale management application 806 generate a seller harvest chart and schedule for a user to complete 846.

On either determining that it is a spot trade 838, or generation of a seller harvest chart and schedule for the user to complete 846, harvest and sale management application 806 may proceed to determine whether all required fields to complete the transaction have been completed. If all required field have not been completed, harvest and sale management application 806 may return to block 836, and proceed therefrom as earlier described. On the other hand, if all required field have been completed, harvest and sale management application 806 may proceed to generate a list of buyers, and quotations/prices 842. The list may be filtered by regions.

Next, harvest and sale management application 806 may display the list for seller to choose the desired offer/quotation. On selection, harvest and sale management application 806 may store a record of the selected harvest/sale transaction in repository 824.

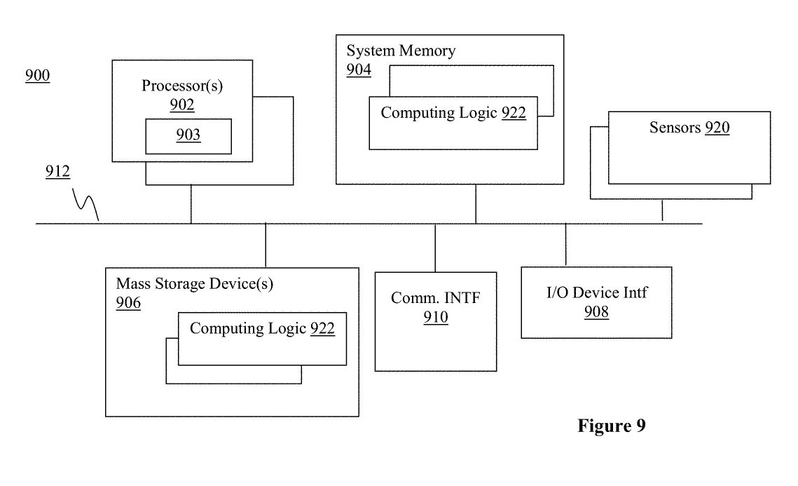

Referring now to FIG. 9, wherein an architecture view of a computer device suitable for practice aspects of a portable device or a cloud server the present disclosure, in accordance with various embodiments, is illustrated. As shown, computer device 900 may include one or more processors 902 and system memory 904. Each processor 902 may include one or more processor cores. In embodiments, a processor 902 may further include hardware accelerator 903 (e.g., constituted with FPGA). System memory 904 may include any known volatile or non-volatile memory.

Additionally, computer device 900 may include a number of sensors 920, e.g., color sensors, GNSS, accelerometer, gyroscope, and so forth. Further, computer device 900 may include mass storage device(s) 906 (such as solid state drives), input/output device interface 908 (to interface with various components, and communication interfaces 910 (such as network interface cards, modems and so forth). In embodiments, communication interfaces 910 may support wired or wireless communication, including near field communication. The elements may be coupled to each other via system bus 912, which may represent one or more buses. In the case of multiple buses, they may be bridged by one or more bus bridges (not shown).

Each of these elements may perform its conventional functions known in the art. In particular, system memory 904 and mass storage device(s) 906 may be employed to store a working copy and a permanent copy of the executable code of the programming instructions implementing the operations described earlier, e.g., but not limited to, operations associated with seed/crop selection, soil management, pesticide and disease control, and harvest/sale, collectively referred to as computing logic 922. The programming instructions may comprise assembler instructions supported by processor(s) 902 or high-level languages, such as, for example, C, that can be compiled into such instructions. In embodiments, system memory 904 and mass storage device(s) 906 may also be employed to store a working copy and a permanent copy of various working or reference data, such as, the repository, calibration or reference data earlier described. In embodiments, some aspects of computing logic 922 may be implemented in hardware accelerator 903.

The permanent copy of the executable code of the programming instructions may be placed into permanent mass storage device(s) 906 in the factory, or in the field, through, for example, a distribution medium (not shown), such as a compact disc (CD), or through communication interface 910 (from a distribution server (not shown)). Similarly, the encoding of hardware accelerator 903 may be performed in the factory or subsequently in the field.

The number, capability and/or capacity of these elements 910-912 may vary, depending on the intended use of example computer device 900, e.g., whether example computer device 900 is used as portable device 110 or cloud server 120 of FIG. 1. The constitutions of these elements 910-912 are otherwise known, and accordingly will not be further described.

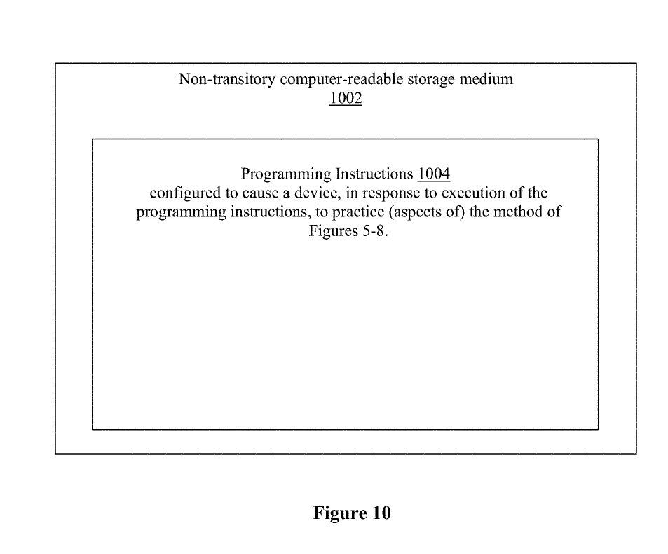

FIG. 10 illustrates an example non-transitory computer-readable storage medium having instructions configured to practice all or selected ones of the operations earlier described, in accordance with various embodiments. As illustrated, non-transitory computer-readable storage medium 1002 may include the executable code of a number of programming instructions (or bit streams for encoding hardware accelerators) 1004. Executable code of programming instructions (or bit streams for encoding hardware accelerators) 1004 may be configured to enable a device, e.g., computer device 900, in response to execution of the executable code/programming instructions (and/or operation of hardware accelerators), to perform, e.g., various operations associated with seed/crop selection, soil management, pesticide and disease control, and harvest/sales, described with references to FIGS. 1-8. In alternate embodiments, executable code/programming instructions (or bit streams for encoding hardware accelerators) 1004 may be disposed on multiple non-transitory computer-readable storage medium 1002 instead. In still other embodiments, executable code/programming instructions (or bit streams for encoding hardware accelerators) 1004 may be encoded in transitory computer readable medium, such as signals.

Referring back to FIG. 9, for one embodiment, at least one of processors 902 may be packaged together with a computer-readable storage medium having some or all of computing logic 922 (in lieu of storing in system memory 904 and/or mass storage device 906) configured to practice all or selected ones of the operations earlier described with references to FIG. 1-8. For one embodiment, at least one of processors 902 may be packaged together with a computer-readable storage medium having some or all of computing logic 922 to form a System in Package (SiP). For one embodiment, at least one of processors 902 may be integrated on the same die with a computer-readable storage medium having some or all of computing logic 922. For one embodiment, at least one of processors 902 may be packaged together with a computer-readable storage medium having some or all of computing logic 922 to form a System on Chip (SoC). For at least one embodiment, the SoC may be utilized in, e.g., but not limited to, a hybrid computing tablet/laptop.

Thus an improved apparatus, method and storage medium associated with spectral signature assisted finger associated user application has been described.

Example 1 may be an apparatus for performing agricultural testing and optimization, comprising: a cavity to receive a container of soil nutrient solution sample of a location in an agricultural region; one or more sensors to collect sensor data from the soil nutrient solution sample; and one or more agricultural testing and optimization applications to perform agricultural testing and optimization for the location, based at least in part on the sensor data collected from the soil nutrient solution sample.

Example 2 may be example 1, wherein the cavity is elongated cylindrical in shape, and the container is similarly elongated and cylindrical in shape, and the cavity and the container are complementarily sized.

Example 3 may be example 1, wherein the one or more sensors comprise one or more red-blue-green (RGB) color sensors, and the apparatus further comprises one or more light-emitting diode (LED) light sources to project light onto RGB color sensors through the soil nutrient solution sample.

Example 4 may be example 3 further comprising a body that defines an interior space to house the cavity, the one or more sensors, and the one or more applications, wherein the body is substantially sealed to prevent ambient light from entering the interior space.

Example 5 may be example 1, wherein the one or more sensors comprises an array of electrochemical sensors disposed on an integrated circuit.

Example 6 may be example 1, wherein the one or more sensors further comprises global network satellite system (GNSS) sensors, gyroscope, magnetometer, or accelerometer.

Example 7 may be example 1, wherein the one or more agricultural testing and optimization applications include a seed/crop recommendation application to recommend seed/crop for the location.

Example 8 may be example 7, further comprising a local repository of prior soil measurements of the agricultural region, a local repository of crops suitable for the agricultural region, a local repository of seeds suitable for the agricultural region, or a local repository of prior seed/crop recommendations for the agricultural region.

Example 9 may be example 1, wherein the one or more agricultural testing and optimization applications include a soil management recommendation application to provide soil management recommendations for crops being grown at the location.

Example 10 may be example 9, further comprising a local repository of optimum soil and crop combinations for the agricultural region.

Example 11 may be example 1, wherein the one or more agricultural testing and optimization applications include a pesticide or additive recommendation application to provide pesticide or additive recommendations for crops being grown at the location.

Example 12 may be example 11, further comprising a local repository of pest and disease diagnostic information, a local repository of known crop problems and associated pesticide and additive treatment information, or a local repository of local suppliers of pesticide and additives.

Example 13 may be example 1, wherein the one or more agricultural testing and optimization applications include a harvest and sale recommendation application to assist a farmer in harvest or sale transactions.

Example 14 may be example 13, further comprising a local repository of crop buyer quotations, or a local repository of harvest/sale transactions.

Example 15 may be example 1, further comprising a processor and memory arrangement to host and operate the one or more agricultural testing and optimization applications.

Example 16 may be example 1, further comprising one or more communication interfaces that includes a cellular communication interface, a WiFi communication interface, a Bluetooth® communication interface, or a near field communication interface to facilitates the one or more agricultural testing and optimization applications in communication with one or more cloud servers.

Example 17 may be example 1, further comprising a touch screen to facilitate user interactions with the one or more agricultural testing and optimization applications.

Example 18 may be any one of examples 1-17, wherein the apparatus is a portable device.

Example 19 may be an apparatus for performing agricultural testing and optimization, comprising: one or more storage devices to store a plurality of repositories of agricultural data for a plurality of agricultural regions; and one or more processor and memory arrangements to host and operate a plurality of cloud/fog data analytics tracking and services to provide agricultural testing and optimization support services to a plurality of portable integrated agricultural testing and optimization devices that operate in a plurality of locations of the plurality of agricultural regions; wherein the plurality of portable integrated agricultural testing and optimization devices with support of the apparatus, provide in field agricultural testing and optimization.

Example 20 may be example 19, wherein the plurality of repositories of agricultural data comprise a cloud/fog repository of optimum soil and crop combination for the various agricultural regions, or a cloud/fog repository of registered seeds.

Example 21 may be example 19, wherein the plurality of repositories of agricultural data comprise a cloud/fog repository of nutrient measurements from multiple sources.

Example 22 may be example 19, wherein the plurality of repositories of agricultural data comprise a cloud/fog repository of crop problems and associated pesticides and additives, or a cloud/fog repository of various suppliers of various pesticides and additives.

Example 23 may be example 19, wherein the plurality of repositories of agricultural data comprise a repository to store buyer quotation information, or a repository to store various harvest/sale transactions.

Example 24 may be a method for performing agricultural testing and optimization, comprising: receiving, by a portable agricultural testing and optimization device, at a location in an agricultural region, a container of soil nutrient solution sample of the location; collecting, by the portable agricultural testing and optimization device, at the location, sensor data from the soil nutrient solution sample; and operating, by the portable agricultural testing and optimization device, at the location, one or more agricultural testing and optimization applications to perform agricultural testing and optimization for the location, based at least in part on the sensor data collected from the soil nutrient solution sample.

Example 25 may be example 24, wherein collecting the sensor data comprises: projecting light, by a light source of the portable agricultural testing and optimization device, onto the soil nutrient solution sample; and sensing the projected light, by red-blue-green (RGB) color sensors of the portable agricultural testing and optimization device, after the projected light passed through the soil nutrient solution sample.

Example 26 may be example 24, wherein collecting the sensor data comprises collecting the sensor data using an integrated array of sensor elements.

Example 27 may be example 24, wherein operating one or more agricultural testing and optimization applications comprise operating a seed/crop recommendation application to recommend seed/crop for the location.

Example 28 may be example 24, wherein operating the seed/crop recommendation application to recommend seed/crop for the location includes operating the seed/crop recommendation application to: determine whether a test pH value for the location is available or use a default pH value for the location; select crops for the location, filtered by land or soil types; and present the selected crops for selection.

Example 29 may be example 28, wherein operating the seed/crop recommendation application to recommend seed/crop for the location further includes operating the seed/crop recommendation application to: generate a list of seeds in response to a selection of a presented crop; receive further inputs on target seed sowing and harvest months; and generate one or more seed recommendations from the list of seeds, based at least in part on the further inputs.

Example 30 may be example 24, wherein operating one or more agricultural testing and optimization applications comprise operating a soil management recommendation application to provide soil management recommendations for crops being grown at the location.

Example 31 may be example 30, wherein operating the soil management recommendation application to provide soil management recommendations for crops being grown at the location includes operating the soil management recommendation application to: receive inputs on nutrient, crop type or soil type; perform signal analysis or nutrient level analysis, on the sensor data, based at least in part on the received inputs; and generate fertilizer recommendations, based at least in part on results of the signal analysis or results of the nutrient analysis.

Example 32 may be example 24, wherein operating one or more agricultural testing and optimization applications comprise operating a pesticide or additive recommendation application to provide pesticide or additive recommendations for crops being grown at the location.

Example 33 may be example 32, wherein operating a pesticide or additive recommendation application to provide pesticide or additive recommendations for crops being grown at the location includes operating a pesticide or additive recommendation application to: receive a crop type; generate a list of possible crop lifecycle stages; generate a list of possible crop symptoms; and generate a crop diagnostic survey.

Example 34 may be example 33, wherein operating the pesticide or additive recommendation application to provide pesticide or additive recommendations for crops being grown at the location further includes operating the pesticide or additive recommendation application to: receive indications of crop symptoms identified from the list; receive answers to the crop diagnostic survey; determine whether soil treatment is to be performed; and recommend crop treatment if no soil treatment is to be performed.

Example 35 may be example 34, wherein operating the pesticide or additive recommendation application to provide pesticide or additive recommendations for crops being grown at the location further includes operating the pesticide or additive recommendation application to: determine whether one or more needed pesticides or additives are available from a local supplier, when soil treatment is to be performed; recommend immediate action, and later treatment with the one or more pesticides and additives, when the one or more needed pesticides or additives are not available from the local supplier; and recommend immediate treatment with the one or more pesticides or additives, wherein the one or more needed pesticides or additives are not available from the local supplier.

Example 36 may be example 24, wherein operating one or more agricultural testing and optimization applications comprise operating a harvest and sale recommendation application to assist a farmer in harvest or sale transactions.

Example 37 may be example 36, wherein operating a harvest and sale recommendation application to assist a farmer in harvest or sale transactions includes operating the harvest and sale recommendation application to receive input on a crop type; determine that a current list of buyers for the crop type is available or generate the current list of buyers for the crop type; present the current list of buyers for the crop type; and receive identifications of one or more buyers selected from the current list of buyers.

Example 38 may be example 37, wherein operating a harvest and sale recommendation application to assist a farmer in harvest or sale transactions further includes operating the harvest and sale recommendation application to: determine whether a transaction with the one or more buyers selected from the current list of buyers is spot trade; solicit and receive user inputs on harvesting date, frequency and duration of harvest, and generate a seller harvest chart or schedule, in response to the user inputs, when the transaction is not a spot trade; and generate a list of buyers, and quotations/prices, on determination the transaction is a spot trade, or on generation of the seller harvest chart or schedule, when the transaction is not a spot trade.

Example 39 may be one or more computer-readable media (CRM) having instructions stored therein to cause a portable agricultural testing and optimization device, in response to execution of the instruction by the portable agricultural testing and optimization device, to: receive at a location in an agricultural region, a container of soil nutrient solution sample of the location; collect at the location, sensor data from the soil nutrient solution sample; and operate at the location, one or more agricultural testing and optimization applications to perform agricultural testing and optimization for the location, based at least in part on the sensor data collected from the soil nutrient solution sample.

Example 40 may be example 39, wherein to collect the sensor data comprises: to project light, with a light source of the portable agricultural testing and optimization device, onto the soil nutrient solution sample; and to sense the projected light, with red-blue-green (RGB) color sensors of the portable agricultural testing and optimization device, after the projected light passed through the soil nutrient solution sample.

Example 41 may be example 39, wherein to collect the sensor data comprises to collect the sensor data from an integrated array of sensor elements.

Example 42 may be example 39, wherein to operate one or more agricultural testing and optimization applications comprises to operate a seed/crop recommendation application to recommend seed/crop for the location.

Example 43 may be example 39, wherein to operate the seed/crop recommendation application to recommend seed/crop for the location includes to operate the seed/crop recommendation application to: determine whether a test pH value for the location is available or use a default pH value for the location; select crops for the location, filtered by land or soil types; and present the selected crops for selection.

Example 44 may be example 42, wherein to operate the seed/crop recommendation application to recommend seed/crop for the location further includes to operate the seed/crop recommendation application to: generate a list of seeds in response to a selection of a presented crop; receive further inputs on target seed sowing and harvest months; and generate one or more seed recommendations from the list of seeds, based at least in part on the further inputs.

Example 45 may be example 39, wherein to operate one or more agricultural testing and optimization applications comprise to operate a soil management recommendation application to provide soil management recommendations for crops being grown at the location.

Example 46 may be example 45, wherein to operate the soil management recommendation application to provide soil management recommendations for crops being grown at the location includes to operate the soil management recommendation application to: receive inputs on nutrient, crop type or soil type; perform signal analysis or nutrient level analysis, on the sensor data, based at least in part on the received inputs; and generate fertilizer recommendations, based at least in part on results of the signal analysis or results of the nutrient analysis.

Example 47 may be example 39, wherein to operate one or more agricultural testing and optimization applications comprise to operate a pesticide or additive recommendation application to provide pesticide or additive recommendations for crops being grown at the location.

Example 48 may be example 47, wherein to operate a pesticide or additive recommendation application to provide pesticide or additive recommendations for crops being grown at the location includes to operate a pesticide or additive recommendation application to: receive a crop type; generate a list of possible crop lifecycle stages; generate a list of possible crop symptoms; and generate a crop diagnostic survey.

Example 49 may be example 48, wherein to operate the pesticide or additive recommendation application to provide pesticide or additive recommendations for crops being grown at the location further includes to operate the pesticide or additive recommendation application to: receive indications of crop symptoms identified from the list; receive answers to the crop diagnostic survey; determine whether soil treatment is to be performed; and recommend crop treatment if no soil treatment is to be performed.

Example 50 may be example 49, wherein to operate the pesticide or additive recommendation application to provide pesticide or additive recommendations for crops being grown at the location further includes to operate the pesticide or additive recommendation application to: determine whether one or more needed pesticides or additives are available from a local supplier, when soil treatment is to be performed; recommend immediate action, and later treatment with the one or more pesticides and additives, when the one or more needed pesticides or additives are not available from the local supplier; and recommend immediate treatment with the one or more pesticides or additives, wherein the one or more needed pesticides or additives are not available from the local supplier.

Example 51 may be example 39, wherein to operate one or more agricultural testing and optimization applications comprise to operate a harvest and sale recommendation application to assist a farmer in harvest or sale transactions.

Example 52 may be example 51, wherein to operate a harvest and sale recommendation application to assist a farmer in harvest or sale transactions includes operating the harvest and sale recommendation application to receive input on a crop type; determine that a current list of buyers for the crop type is available or generate the current list of buyers for the crop type; present the current list of buyers for the crop type; and receive identifications of one or more buyers selected from the current list of buyers.

Example 53 may be example 52, wherein to operate a harvest and sale recommendation application to assist a farmer in harvest or sale transactions further includes to operate the harvest and sale recommendation application to: determine whether a transaction with the one or more buyers selected from the current list of buyers is spot trade; solicit and receive user inputs on harvesting date, frequency and duration of harvest, and generate a seller harvest chart or schedule, in response to the user inputs, when the transaction is not a spot trade; and generate a list of buyers, and quotations/prices, on determination the transaction is a spot trade, or on generation of the seller harvest chart or schedule, when the transaction is not a spot trade.

Example 54 may be an apparatus for performing agricultural testing and optimization, comprising: means for receiving a container of soil nutrient solution sample of a location in an agricultural region; means for collecting sensor data from the soil nutrient solution sample; and means for performing agricultural testing and optimization for the location, based at least in part on the sensor data collected from the soil nutrient solution sample.

Example 55 may be an apparatus for performing agricultural testing and optimization, comprising: means for storing a plurality of repositories of agricultural data for a plurality of agricultural regions; and means for hosting and operating a plurality of cloud/fog data analytics tracking and services to provide agricultural testing and optimization support services to a plurality of portable integrated agricultural testing and optimization devices that operate in a plurality of locations of the plurality of agricultural regions; wherein the plurality of portable integrated agricultural testing and optimization devices, with support of the apparatus, provide in field agricultural testing and optimization.

Although certain embodiments have been illustrated and described herein for purposes of description, a wide variety of alternate and/or equivalent embodiments or implementations calculated to achieve the same purposes may be substituted for the embodiments shown and described without departing from the scope of the present disclosure. This application is intended to cover any adaptations or variations of the embodiments discussed herein. Therefore, it is manifestly intended that embodiments described herein be limited only by the claims.

Where the disclosure recites “a” or “a first” element or the equivalent thereof, such disclosure includes one or more such elements, neither requiring nor excluding two or more such elements. Further, ordinal indicators (e.g., first, second or third) for identified elements are used to distinguish between the elements, and do not indicate or imply a required or limited number of such elements, nor do they indicate a particular position or order of such elements unless otherwise specifically stated.

What is claimed is:

1. An apparatus for performing agricultural testing and optimization, comprising:
a cavity to receive a container of soil nutrient solution sample of a location in an agricultural region;
one or more sensors to collect sensor data from the soil nutrient solution sample; and
one or more agricultural testing and optimization applications to perform agricultural testing and optimization for the location, based at least in part on the sensor data collected from the soil nutrient solution sample.

2. The apparatus of claim 1, wherein the cavity is elongated cylindrical in shape, and the container is similarly elongated and cylindrical in shape, and the cavity and the container are complementarily sized.

3. The apparatus of claim 1, wherein the one or more sensors comprise one or more red-blue-green (RGB) color sensors, and the apparatus further comprises one or more light-emitting diode (LED) light sources to project light onto RGB color sensors through the soil nutrient solution sample.

4. The apparatus of claim 3 further comprising a body that defines an interior space to house the cavity, the one or more sensors, and the one or more applications, wherein the body is substantially sealed to prevent ambient light from entering the interior space.

5. The apparatus of claim 1, wherein the one or more sensors comprises an array of electrochemical sensors disposed on an integrated circuit.

6. The apparatus of claim 1, wherein the one or more sensors further comprises global network satellite system (GNSS) sensors, gyroscope, magnetometer, or accelerometer.

7. The apparatus of claim 1, wherein the one or more agricultural testing and optimization applications include a seed/crop recommendation application to recommend seed/crop for the location, and the apparatus further comprises a local repository of prior soil measurements of the agricultural region, a local repository of crops suitable for the agricultural region, a local repository of seeds suitable for the agricultural region, or a local repository of prior seed/crop recommendations for the agricultural region.

8. The apparatus of claim 1, wherein the one or more agricultural testing and optimization applications include a soil management recommendation application to provide soil management recommendations for crops being grown at the location, and the apparatus further comprises a local repository of optimum soil and crop combinations for the agricultural region.

9. The apparatus of claim 1, wherein the one or more agricultural testing and optimization applications include a pesticide or additive recommendation application to provide pesticide or additive recommendations for crops being grown at the location, and the apparatus further comprises a local repository of pest and disease diagnostic information, a local repository of known crop problems and associated pesticide and additive treatment information, or a local repository of local suppliers of pesticide and additives.

10. The apparatus of claim 1, wherein the one or more agricultural testing and optimization applications include a harvest and sale recommendation application to assist a farmer in harvest or sale transactions, and the apparatus further comprises a local repository of crop buyer quotations, or a local repository of harvest/sale transactions.

11. The apparatus of claim 1, further comprising
a processor and memory arrangement to host and operate the one or more agricultural testing and optimization applications;
one or more communication interfaces that includes a cellular communication interface, a WiFi communication interface, a Bluetooth® communication interface, or a near field communication interface to facilitates the one or more agricultural testing and optimization applications in communication with one or more cloud servers; and
a touch screen to facilitate user interactions with the one or more agricultural testing and optimization applications.

12. The apparatus of claim 1, wherein the apparatus is a portable device.

13. An apparatus for performing agricultural testing and optimization, comprising:
one or more storage devices to store a plurality of repositories of agricultural data for a plurality of agricultural regions; and
one or more processor and memory arrangements to host and operate a plurality of cloud/fog data analytics tracking and services to provide agricultural testing and optimization support services to a plurality of portable integrated agricultural testing and optimization devices that operate in a plurality of locations of the plurality of agricultural regions;
wherein the plurality of portable integrated agricultural testing and optimization devices with support of the apparatus, provide in field agricultural testing and optimization.

14. The apparatus of claim 13, wherein the plurality of repositories of agricultural data comprise a cloud/fog repository of optimum soil and crop combination for the various agricultural regions, a cloud/fog repository of registered seeds, a cloud/fog repository of nutrient measurements from multiple sources, a cloud/fog repository of crop problems and associated pesticides and additives, a cloud/fog repository of various suppliers of various pesticides and additives, or a repository to store buyer quotation information, or a repository to store various harvest/sale transactions.

15. A method for performing agricultural testing and optimization, comprising:
receiving, by a portable agricultural testing and optimization device, at a location in an agricultural region, a container of soil nutrient solution sample of the location;
collecting, by the portable agricultural testing and optimization device, at the location, sensor data from the soil nutrient solution sample; and
operating, by the portable agricultural testing and optimization device, at the location, one or more agricultural testing and optimization applications to perform agricultural testing and optimization for the location, based at least in part on the sensor data collected from the soil nutrient solution sample.

16. The method of claim 15, wherein operating one or more agricultural testing and optimization applications comprise operating a seed/crop recommendation application to recommend seed/crop for the location, a soil management recommendation application to provide soil management recommendations for crops being grown at the location, a pesticide or additive recommendation application to provide pesticide or additive recommendations for crops being grown at the location, or a harvest and sale recommendation application to assist a farmer in harvest or sale transactions.

17. One or more computer-readable media (CRM) having instructions stored therein to cause a portable agricultural testing and optimization device, in response to execution of the instruction by the portable agricultural testing and optimization device, to:
receive at a location in an agricultural region, a container of soil nutrient solution sample of the location;
collect at the location, sensor data from the soil nutrient solution sample; and
operate at the location, one or more agricultural testing and optimization applications to perform agricultural testing and optimization for the location, based at least in part on the sensor data collected from the soil nutrient solution sample.

18. The CRM of claim 17, wherein to operate one or more agricultural testing and optimization applications comprises to operate a seed/crop recommendation application to recommend seed/crop for the location;
wherein to operate the seed/crop recommendation application to recommend seed/crop for the location includes to operate the seed/crop recommendation application to:
determine whether a test pH value for the location is available or use a default pH value for the location;
select crops for the location, filtered by land or soil types; and
present the selected crops for selection.

19. The CRM of claim 18, wherein to operate the seed/crop recommendation application to recommend seed/crop for the location further includes to operate the seed/crop recommendation application to:
generate a list of seeds in response to a selection of a presented crop;
receive further inputs on target seed sowing and harvest months; and
generate one or more seed recommendations from the list of seeds, based at least in part on the further inputs.

20. The CRM of claim 17, wherein to operate one or more agricultural testing and optimization applications comprise to operate a soil management recommendation application to provide soil management recommendations for crops being grown at the location;
wherein to operate the soil management recommendation application to provide soil management recommendations for crops being grown at the location includes to operate the soil management recommendation application to:
receive inputs on nutrient, crop type or soil type;
perform signal analysis or nutrient level analysis, on the sensor data, based at least in part on the received inputs; and
generate fertilizer recommendations, based at least in part on results of the signal analysis or results of the nutrient analysis.

21. The CRM of claim 17, wherein to operate one or more agricultural testing and optimization applications comprise to operate a pesticide or additive recommendation application to provide pesticide or additive recommendations for crops being grown at the location;
wherein to operate a pesticide or additive recommendation application to provide pesticide or additive recommendations for crops being grown at the location includes to operate a pesticide or additive recommendation application to:
receive a crop type;
generate a list of possible crop lifecycle stages;
generate a list of possible crop symptoms; and
generate a crop diagnostic survey.

22. The CRM of claim 21, wherein to operate the pesticide or additive recommendation application to provide pesticide or additive recommendations for crops being grown at the location further includes to operate the pesticide or additive recommendation application to:
receive indications of crop symptoms identified from the list;
receive answers to the crop diagnostic survey;
determine whether soil treatment is to be performed; and
recommend crop treatment if no soil treatment is to be performed.

23. The CRM of claim 21, wherein to operate the pesticide or additive recommendation application to provide pesticide or additive recommendations for crops being grown at the location further includes to operate the pesticide or additive recommendation application to:
determine whether one or more needed pesticides or additives are available from a local supplier, when soil treatment is to be performed;
recommend immediate action, and later treatment with the one or more pesticides and additives, when the one or more needed pesticides or additives are not available from the local supplier; and
recommend immediate treatment with the one or more pesticides or additives, wherein the one or more needed pesticides or additives are not available from the local supplier.

24. The CRM of claim 17, wherein to operate one or more agricultural testing and optimization applications comprise to operate a harvest and sale recommendation application to assist a farmer in harvest or sale transactions;
wherein to operate a harvest and sale recommendation application to assist a farmer in harvest or sale transactions includes operating the harvest and sale recommendation application to
receive input on a crop type;
determine that a current list of buyers for the crop type is available or generate the current list of buyers for the crop type;
present the current list of buyers for the crop type; and
receive identifications of one or more buyers selected from the current list of buyers.

25. The CRM of claim 24, wherein to operate a harvest and sale recommendation application to assist a farmer in harvest or sale transactions further includes to operate the harvest and sale recommendation application to:
determine whether a transaction with the one or more buyers selected from the current list of buyers is spot trade;
solicit and receive user inputs on harvesting date, frequency and duration of harvest, and generate a seller harvest chart or schedule, in response to the user inputs, when the transaction is not a spot trade; and
generate a list of buyers, and quotations/prices, on determination the transaction is a spot trade, or on generation of the seller harvest chart or schedule, when the transaction is not a spot trade.
